(19) United States
(12) Patent Application Publication
(10) Pub. No.: US 2025/0340716 A1
(43) Pub. Date: Nov. 6, 2025

(54) POLYMER COMPOSITE EMITTING NEAR INFRARED RAY AND NEAR INFRARED EMITTING COMMODITIES COMPRISING THE SAME

(71) Applicant: KYUNGPOOK NATIONAL UNIVERSITY INDUSTRY-ACADEMIC COOPERATION FOUNDATION, Daegu (KR)

(72) Inventor: Jin Gu KIM, Daegu (KR)

(21) Appl. No.: 19/197,078

(22) Filed: May 2, 2025

(30) Foreign Application Priority Data
May 3, 2024 (KR) 10-2024-0059064

(51) Int. Cl. C08K 3/22; C08G 63/183; C09K 11/77

(52) U.S. Cl. CPC C08K 3/22; C08G 63/183; C09K 11/7701; C08K 2003/221; C08K 2201/003; C08K 2201/014

(57) ABSTRACT

The present disclosure relates to a near-infrared ray emitting polymer composite, and more specifically to a near-infrared ray emitting polymer composite, which can emit visible light and near-infrared light at a specific wavelength range, has a high peak intensity in a specific visible light and near-infrared wavelength range, does not cause single yarns during fiber formation, can provide excellent durability of commodities, and can increase the storage period of food when applied to a food container, and near-infrared ray emitting commodities including the same.

### BACKGROUND

### 1. Technical Field

The present disclosure relates to a near-infrared ray emitting polymer composite, and more specifically to a near-infrared ray emitting polymer composite, which can emit visible light and near-infrared light at a specific wavelength range, has a high peak intensity in a specific visible light and near-infrared wavelength range, does not cause single yarns during fiber formation, can provide excellent durability of commodities, and can increase the storage period of food when applied to a food container, and near-infrared ray emitting commodities including the same.

### 2. Related Art

Infrared rays have a longer wavelength than visible light, and are electromagnetic waves belonging to a range from 0.75 μm to 1 mm. When it is classified by wavelength range, those with a wavelength of 0.75 to 3 μm are called near-infrared rays, those with a wavelength of 3 to 25 μm are simply called infrared rays, and those with a wavelength of 25 μm or more are called far-infrared rays.

Among these, near-infrared rays, which have the shortest wavelength, have a stronger thermal effect than visible light or ultraviolet rays, and have the characteristic of transmitting heat only to objects without heating the air, and have high and deep penetration into living tissues, and thus, they are used in various fields such as medical fields related to the human body, as well as industrial purposes for disinfection or sterilization.

The efficacy of this near-infrared ray treatment varies depending on the wavelength, and it can exhibit effects such as treating joints and muscles, enhancing immunity, relieving pain, improving blood circulation and the like.

Meanwhile, in the case of conventional near-infrared ray emitting polymers, there was a problem in that they could not simultaneously exhibit the effects of being able to emit near-infrared rays in a specific wavelength range, not causing single yarns during fiber formation, having excellent durability of a product, and extending the storage period of food when applied to food containers. In particular, even if they could emit visible light and near-infrared rays in a specific wavelength range, there was a problem in that the peak intensity was not high in the specified visible light and near-infrared wavelength range.

Accordingly, there is an urgent need to develop a polymer composite that can emit visible light and near-infrared rays in a specific wavelength range, has high peak intensity in the specified visible light and near-infrared wavelength range, does not cause single yarns during fiber formation, has excellent durability of a product, and extends the storage period of food when applied to food containers.

### SUMMARY

The present disclosure has been devised to solve the above-described problems, and an object of the present disclosure is to provide a near-infrared ray emitting polymer composite, which can emit visible light and near-infrared light in a specific wavelength range, has a high peak intensity in a specific visible light and near-infrared wavelength range, does not cause single yarns during fiber formation, can provide excellent durability of a product, and can increase the storage period of food when applied to a food container, and near-infrared ray emitting commodities including the same

In order to solve the above-described problems, the present disclosure provides a near-infrared ray emitting polymer composite, including a near-infrared ray emitting metal oxide including Nd₂O₃, Er₂O₃, Sm₂O₃ and Pr₂O₃; a near-infrared ray emitting carbon material; and a polymer resin in which the metal oxide and the carbon material are dispersed.

According to an embodiment of the present disclosure, the carbon material may include graphite.

In addition, the carbon material may have an average particle diameter of 25 to 160 nm.

In addition, the metal oxide and carbon material may be included in a total of 1 to 8 wt % of the total weight of the near-infrared ray emitting polymer composite.

In addition, the polymer composite may include 10 to 40 parts by weight of the carbon material based on 100 parts by weight of the metal oxide.

In addition, the polymer composite may include 67 to 88 wt % of Nd₂O₃ and Er₂O₃ based on the total weight of the metal oxide.

In addition, the polymer composite may include 12 to 33 wt % of Sm₂O₃ and Pr₂O₃ based on the total weight of the metal oxide.

In addition, the metal oxide may have an average particle diameter of 50 to 500 nm.

In addition, the polymer resin may include at least one selected from the group consisting of polyamide, polyester, polyketone, liquid crystal polymer, polyolefin, polyphenylene sulfide (PPS), polyether ether ketone (PEEK), polyphenylene oxide (PPO), polyether sulfone (PES), polyether imide (PEI) and polyimide.

In addition, the present disclosure provides a near-infrared ray emitting commodity, including the above-described polymer composite.

According to an embodiment of the present disclosure, the near-infrared ray emitting commodity may include at least one of clothing, a food container and a cosmetic container.

In addition, the near-infrared ray emitting commodity may be formed by the near-infrared ray emitting polymer composite, or is formed by coating the near-infrared ray emitting polymer composite on a predetermined commodity.

The near-infrared ray emitting polymer composite according to the present disclosure and the near-infrared ray emitting commodities including the same can simultaneously exhibit the effects of emitting visible light and near-infrared light in a specific wavelength range, having high peak intensity in a specific visible light and near-infrared wavelength range, not causing single yarns during fiber formation, having excellent durability of a product, and increasing the storage period of food when applied to a food container.

### DETAILED DESCRIPTION

Hereinafter, embodiments of the present disclosure will be described in detail so that those skilled in the art can easily practice the present disclosure. The present disclosure may be implemented in various different forms and is not limited to the embodiments described herein.

The near-infrared ray emitting polymer composite according to an embodiment of the present disclosure is implemented by including a near-infrared ray emitting metal oxide including Nd₂O₃, Er₂O₃, Sm₂O₃ and Pr₂O₃, a near-infrared ray emitting carbon material, and a polymer resin in which the metal oxide and carbon material are dispersed.

First of all, the metal oxide will be described.

As described above, the metal oxide includes Nd₂O₃, Er₂O₃, Sm₂O₃ and Pr₂O₃.

In this case, the Nd₂O₃, Er₂O₃, Sm₂O₃ and Pr₂O₃ perform a function of emitting visible light and near-infrared light in a wavelength range of 600 to 900 nm.

The near-infrared ray emitting metal oxide may include Nd₂O₃ and Er₂O₃ in a total of 67 to 88 wt %, and preferably 70 to 85 wt %. If the Nd₂O₃ and Er₂O₃ are included in a total of less than 67 wt %, visible light and near-infrared light with a wavelength of 600 to 900 nm may not be emitted at the desired level, and if the Nd₂O₃ and Er₂O₃ are included in a total of more than 88 wt %, the wavelength range of the emitted visible light and near-infrared light becomes excessively narrow, and the intensity of the emitted visible light and near-infrared light also decreases.

In addition, the Sm₂O₃ and Pr₂O₃ perform a function of expanding the wavelength range of the emitted visible light and near-infrared light.

The near-infrared ray emitting metal oxide may include Sm₂O₃ and Pr₂O₃ in a total of 12 to 33 wt %, and preferably 15 to 30 wt %. If the Sm₂O₃ and Pr₂O₃ are included in less than 12 wt % in total, the wavelength range of the emitted visible light and near-infrared light may become excessively narrow, and it may cause problems in which the intensity of the emitted visible light and near-infrared light also decreases. If the Sm₂O₃ and Pr₂O₃ are included in more than 33 wt % in total, the visible light and near-infrared light with a wavelength of 600 to 900 nm may not be emitted at the desired level, and the intensity of the visible light and near-infrared light with a wavelength of 600 to 900 nm may decrease.

Meanwhile, the metal oxide according to the present disclosure may have an average particle diameter of 50 to 500 nm, and preferably an average particle diameter of 55 to 490 nm. If the average particle diameter of the metal oxide is less than 50 nm, as metal oxides may be generated on the surface, the desired level of visible light and near-infrared emission characteristics may not be achieved, and it is undesirable in terms of cost. If the average particle diameter exceeds 500 nm, the metal oxide may be present on the surface, which may result in poor surface characteristics, increased generation of single yarns during fiber formation, or decreased durability of the polymer molded body.

Next, the near-infrared ray emitting carbon material will be described.

The near-infrared ray emitting carbon material performs a function of emitting visible light and near-infrared rays in a wavelength range of 500 to 900 nm, and a function of enhancing the peak intensity of visible light and near-infrared rays in the corresponding wavelength range. In particular, when it is used together with the above-described near-infrared ray emitting metal oxide, it may exhibit a synergistic effect of further enhancing the peak intensity of visible light and near-infrared rays in a wavelength range of 500 to 900 nm. Furthermore, when it is used together with a near-infrared ray emitting metal oxide including Nd₂O₃, Er₂O₃, Sm₂O₃ and Pr₂O₃, it may exhibit a synergistic effect of further enhancing the peak intensity of visible light and near-infrared emission in a wavelength range of 500 to 900 nm.

In addition, the carbon material may be used without limitation as long as it is a known carbon material, but preferably, it may be more advantageous to include graphite in terms of exhibiting a synergistic effect of enhancing the emission peak intensity of visible light and near-infrared rays in a wavelength range of 500 to 900 nm while emitting visible light and near-infrared rays in a wavelength range of 500 to 900 nm as described above.

In addition, the carbon material may have an average particle diameter of 25 to 160 nm, and preferably, an average particle diameter of 30 to 150 nm. If the average particle diameter of the carbon material is less than 25 nm, as a carbon material may be generated on the surface, it may not exhibit the desired level of visible light and near-infrared emission characteristics, and it is not desirable in terms of cost. In addition, if the average particle diameter exceeds 160 nm, the surface properties may be poor because there may be metal oxides protruding on the surface, and the generation of single yarns during fiber formation may increase or the durability of the polymer molded body may decrease. In this case, if the carbon material satisfies the above-described average particle diameter range, it may be more advantageous in terms of expressing a synergistic effect of enhancing the emission peak intensity of visible light and near-infrared rays in a wavelength range of 500 to 900 nm while emitting visible light and near-infrared rays in a wavelength range of 500 to 900 nm as described above.

In addition, the carbon material may be included in an amount of 10 to 40 parts by weight based on 100 parts by weight of the metal oxide, and preferably, the carbon material may be included in an amount of 12 to 38 parts by weight based on 100 parts by weight of the metal oxide. If the carbon material is less than 10 parts by weight based on 100 parts by weight of the metal oxide, the emission peak intensity of visible light and near-infrared rays in a wavelength range of 500 to 900 nm may be low, and if the carbon material is more than 40 parts by weight based on 100 parts by weight of the metal oxide, there may be a problem in that the wavelength range of 500 to 650 nm may decrease, and only the wavelength range of 800 to 900 nm may increase.

Next, the polymer resin will be described.

The polymer resin performs a function of accommodating the metal oxide and carbon material such that the metal oxide and carbon material described above are provided by being dispersed.

The polymer resin may be used without limitation as long as it supports the metal oxide and carbon material and does not inhibit visible light and near-infrared ray emission, and preferably may include at least any one selected from the group consisting of polyamide, polyester, polyketone, liquid crystal polymer, polyolefin, polyphenylene sulfide (PPS), polyether ether ketone (PEEK), polyphenylene oxide (PPO), polyether sulfone (PES), polyether imide (PEI) and polyimide, more preferably may include at least any one of polyester and polyolefin, and even more preferably may include at least any one of polyethylene terephthalate (PET) and polypropylene (PP).

For example, the polyamide may be a known polyamide compound such as nylon 6, nylon 66, nylon 11, nylon 610, nylon 12, nylon 46, nylon 9T (PA-9T), kiana and aramid.

In addition, as another example, the polyester may be a known polyester compound such as polyethylene terephthalate (PET), polytrimethylene terephthalate (PTT), polybutylene terephthalate (PBT) and polycarbonate.

In addition, as still another example, the polyolefin may be a known polyolefin compound such as polyethylene, polypropylene, polystyrene, polyisobutylene and ethylene vinyl alcohol.

The liquid crystal polymer may be used without limitation as long as it is a polymer that exhibits liquid crystal properties in a solution or dissolved state, and may be a known type, and thus, the present disclosure is not particularly limited thereto.

Meanwhile, the near-infrared ray emitting polymer composite according to the present disclosure may include the metal oxide and carbon material in a total of 1 to 8 wt % of the total weight of the near-infrared ray emitting polymer composite, and preferably, it may include the metal oxide and carbon material in a total of 2 to 7.5 wt % of the total weight of the near-infrared ray emitting polymer composite. If the metal oxide and carbon material are included in a total of less than 1 wt % of the total weight of the near-infrared ray emitting polymer composite, visible light and near-infrared rays may not be emitted at the desired level, and the emission peak intensities of visible light and near-infrared light may be reduced, and if the metal oxide and carbon material are included in a total of more than 8 wt % of the total weight of the near-infrared ray emitting polymer composite, there may be problem in that single yarns are formed during fiber formation or the durability of the product is reduced.

In addition, the present disclosure provides a near-infrared ray emitting commodity including the above-described near-infrared ray emitting polymer composite according to the present disclosure.

The above-mentioned near-infrared ray emitting commodity may be formed by the near-infrared ray emitting polymer composite, or may be formed by coating a predetermined commodity with the near-infrared ray emitting polymer composite.

As an example in which the near-infrared ray emitting commodity is formed by the near-infrared ray emitting polymer composite, a near-infrared ray emitting fiber may be formed through the near-infrared ray emitting polymer composite, and in this case, the near-infrared ray emitting fiber may be manufactured by spinning the near-infrared ray emitting polymer composite, but is not limited thereto.

In this case, by spinning the near-infrared ray emitting polymer composite, a near-infrared ray emitting non-woven fabric including a plurality of the near-infrared ray emitting fibers may be manufactured. Alternatively, clothing may be manufactured through the fiber formed by spinning the near-infrared ray emitting polymer composite.

In addition, as another example in which the near-infrared ray emitting commodity is formed of the near-infrared ray emitting polymer composite, the near-infrared ray emitting polymer composite may be injected to form food containers, cosmetic containers and the like, and in this case, the food containers and cosmetic containers themselves may be formed of the near-infrared ray emitting polymer composite, or only some parts or areas of the food containers and cosmetic containers may be formed by using the near-infrared ray emitting polymer composite described above.

In addition, as an example in which the near-infrared ray emitting commodity is formed by coating the near-infrared ray emitting polymer composite on a predetermined commodity, the near-infrared ray emitting polymer composite may be coated on a predetermined fiber to manufacture a near-infrared ray emitting fiber, and clothing may be manufactured by using the near-infrared ray emitting fiber. Alternatively, the near-infrared ray emitting polymer composite may be coated on a predetermined fabric to manufacture a near-infrared ray emitting fabric, and clothing may be manufactured by using the near-infrared ray emitting fabric. Alternatively, the near-infrared ray emitting polymer composite may be coated on predetermined clothing to manufacture a near-infrared ray emitting clothing.

In addition, as another example in which the near-infrared ray emitting commodity is formed by coating the near-infrared ray emitting polymer composite on a predetermined commodity, the near-infrared ray emitting polymer composite may be coated on a predetermined food container or cosmetic container to manufacture a near-infrared ray emitting food container or a near-infrared ray emitting cosmetic container. In this case, the near-infrared ray emitting polymer composite may be coated on only a part of the above-defined food container or cosmetic container, or the near-infrared ray emitting polymer composite may be coated on the entire food container or cosmetic container, and thus, the present disclosure does not specifically limit the same.

Meanwhile, the commodity may include at least any one of clothing, a food container and a cosmetic container as described above. However, it is not limited thereto, and since it can be applied to various fields, it is not limited to the commodities.

In this case, if the commodity is clothing, the near-infrared ray emitting commodity may be a near-infrared ray emitting commodity, and the near-infrared ray emitting commodity may exhibit effects such as the treatment of joints and muscles, the enhancement of immunity, pain relief and the improvement of blood circulation.

In addition, if the commodity is a food container or a cosmetic container, the near-infrared ray emitting commodity may be a near-infrared ray emitting food container or a near-infrared ray emitting cosmetic container, and the near-infrared ray emitting food container may improve the storage period of stored food, and the near-infrared ray emitting cosmetic container may improve the storage period of stored cosmetics.

Meanwhile, the near-infrared ray emitting polymer composite according to the present disclosure and the near-infrared ray emitting commodity including the same may emit visible light and near-infrared rays in a specific wavelength range, have high peak intensities in a specific visible light and near-infrared wavelength range, do not cause single yarns during fiber formation, have excellent durability of the article, and may exhibit effects such as increasing the storage period of food when it is applied to a food container.

The present disclosure will be described more specifically through the following examples, but the following examples do not limit the scope of the present disclosure, and should be interpreted as helping to understand the present disclosure.

### EXAMPLES

### Example 1

A near-infrared ray emitting polymer composite was prepared by mixing 95 wt % of polyethylene terephthalate (PET) as a polymer resin and 5 wt % of a metal oxide and a carbon material. In this case, the metal oxide included 75 wt % of Nd₂O₃ and Er₂O₃ at a weight ratio of 2:1 and 25 wt % of Sm₂O₃ and Pr₂O₃ at a weight ratio of 1:1 based on the total weight of the metal oxide, and the carbon material was prepared to be included in an amount of 25 wt % based on 100 wt % of the metal oxide. In this case, the total average particle diameter of the metal oxide was 275 nm, and the carbon material was graphite having an average particle diameter of 90 nm.

### Examples 2 to 9 and Comparative Example

These were prepared in the same manner as Example 1, except that the total content of metal oxide and carbon material, the content of carbon material, the average particle diameter of carbon material, the average particle diameter of metal oxide, and whether a carbon material was included were changed to prepare near-infrared ray emitting polymer composites as shown in Tables 1 and 2 below.

### Experimental Example 1: Evaluation of Visible Light and Near-Infrared Ray Emission

The visible light and near-infrared ray emitting polymer composites according to the examples and comparative example were evaluated for visible light and near-infrared ray emission.

Specifically, the near-infrared ray emitting polymer composites were excited with light at wavelengths of 514 nm, 633 nm and 785 nm, respectively, and the visible light and near-infrared rays emitted at wavelengths of 500 to 1,000 nm were measured for each section. In this case, the near-infrared intensity in each section (Section 1 500 nm to less than 600 nm, Section 2 600 nm to less than 700 nm, Section 3 700 nm to less than 800 nm, Section 4 800 nm to less than 900 nm, and Section 5 900 nm to 1,000 nm) is shown in Tables 1 and 2 below, based on Example 1 as 100.

### Experimental Example 2: Evaluation of Prevention of Generation of Single Yarns

The near-infrared ray emitting polymer composites according to the examples and comparative example were spun by melt spinning to form near-infrared ray emitting fibers having an average fiber diameter of 25 μm. In the case where the generation of single yarns did not occur during the spinning process, it was marked as-o, and in the case where the generation of single yarns occurred, it was marked as-x, and the prevention of the generation of single yarns was evaluated and shown in Tables 1 and 2 below.

### Experimental Example 3: Evaluation of Durability

The tensile strength of the near-infrared ray emitting polymer composites according to the examples and comparative example was measured at a speed of 5 mm/min by using a universal test machine (Universal test machine, ZWICK-Z50, GERMANY). The tensile strength of Example 1 was set as 100, and the relative ratio of the tensile strengths of the other examples and comparative examples was measured to evaluate the durability, which is shown in Tables 1 and 2 below.

TABLE 1

| | | | Example | Example | Example | Example | Example |
|---|---|---|---|---|---|---|---|
| Classification | | | 1 | 2 | 3 | 4 | 5 |
| Near- | Metal Oxide | Total Content of | 75 | 75 | 75 | 75 | 75 |
| infrared ray | Emitting | Nd₂O₃ And Er₂O₃ | | | | | |
| emitting | Near-infrared | (wt %) | | | | | |
| polymer | Ray | Total Content of | 25 | 25 | 25 | 25 | 25 |
| composite | | Sm₂O₃ And Pr₂O₃ | | | | | |
| | | (wt %) | | | | | |
| | | Total Average | 275 | 275 | 275 | 275 | 275 |
| | | Particle Diameter | | | | | |
| | | (nm) | | | | | |
| | Carbon | Content of | 25 | 25 | 25 | 5 | 50 |
| | Material | Carbon Material | | | | | |
| | Emitting | (parts by weight) | | | | | |
| | Near-infrared | Average Particle | 90 | 90 | 90 | 90 | 90 |
| | Ray | Diameter (nm) | | | | | |
| | Total Content of Metal Oxide | | 5 | 0.5 | 10 | 5 | 5 |
| | And Carbon Material (wt %) | | | | | | |
| Evaluation | Section 1 | 500 nm to less | 100 | 60 | 103 | 98 | 24 |
| | | than 600 nm | | | | | |
| | Section 2 | 600 nm to less | 100 | 54 | 104 | 76 | 43 |
| | | than 700 nm | | | | | |
| | Section 3 | 700 nm to less | 100 | 56 | 105 | 82 | 96 |
| | | than 800 nm | | | | | |
| | Section 4 | 800 nm to less | 100 | 53 | 106 | 80 | 116 |
| | | than 900 nm | | | | | |
| | Section 5 | 900 nm to | 100 | 55 | 103 | 92 | 98 |
| | | 1,000 nm | | | | | |

| | | | | | |
|---|---|---|---|---|---|
| Evaluation of Prevention of | ○ | ○ | × | ○ | ○ |
| Single yarn Generation | | | | | |
| Evaluation of Durability | 100 | 104 | 94 | 104 | 97 |

TABLE 2

| | | | Example | Example | Example | Example | Comparative |
|---|---|---|---|---|---|---|---|
| Classification | | | 6 | 7 | 8 | 9 | Example |
| Near- | Metal Oxide | Total Content of | 75 | 75 | 75 | 75 | 75 |
| infrared | Emitting | Nd₂O₃ And Er₂O₃ | | | | | |
| ray | Near-infrared | (wt %) | | | | | |
| emitting | Ray | Total Content of | 25 | 25 | 25 | 25 | 25 |
| polymer | | Sm₂O₃ And Pr₂O₃ | | | | | |
| composite | | (wt %) | | | | | |
| | | Total Average | 275 | 275 | 40 | 550 | 275 |
| | | Particle Diameter | | | | | |
| | | (nm) | | | | | |
| | Carbon | Content of | 25 | 25 | 25 | 25 | — |
| | Material | Carbon Material | | | | | |
| | Emitting | (parts by weight) | | | | | |
| | Near-infrared | Average Particle | 20 | 200 | 90 | 90 | — |
| | Ray | Diameter (nm) | | | | | |
| | Total Content of Metal Oxide | | 5 | 5 | 5 | 5 | 4 |
| | And Carbon Material (wt %) | | | | | | |
| Evaluation | Section 1 | 500 nm to less | 71 | 99 | 80 | 91 | 88 |
| | | than 600 nm | | | | | |
| | Section 2 | 600 nm to less | 73 | 94 | 79 | 86 | 37 |
| | | than 700 nm | | | | | |
| | Section 3 | 700 nm to less | 72 | 96 | 80 | 90 | 52 |
| | | than 800 nm | | | | | |
| | Section 4 | 800 nm to less | 73 | 92 | 81 | 82 | 64 |
| | | than 900 nm | | | | | |
| | Section 5 | 900 nm to | 74 | 93 | 83 | 88 | 81 |
| | | 1,000 nm | | | | | |

| | | | | | |
|---|---|---|---|---|---|
| Evaluation of Prevention of | ○ | × | ○ | × | ○ |
| Single yarn Generation | | | | | |
| Evaluation of Durability | 102 | 67 | 103 | 54 | 102 |

As can be seen from Tables 1 and 2 above, Example 1, which satisfied all of the total content of metal oxide and carbon material, the content of carbon material, the average particle diameter of carbon material, the average particle diameter of metal oxide and whether the carbon material is included, could simultaneously exhibit a wide wavelength range of near-infrared rays emitted, could emit near-infrared rays with a wavelength of 600 to 900 nm at high intensity, had excellent durability, and exhibited the effect of not causing single yarns during fiber formation, compared to Examples 2 to 9 and Comparative Examples, which omitted at least one of the above.

Although an embodiment of the present disclosure has been described above, the spirit of the present disclosure is not limited to the embodiments presented in this specification, and those skilled in the art who understand the spirit of the present disclosure will be able to easily propose other embodiments by modifying, changing, deleting or adding components within the scope of the same spirit, but this will also fall within the spirit scope of the present disclosure.

1. A near-infrared ray emitting polymer composite, comprising:
a near-infrared ray emitting metal oxide comprising Nd₂O₃, Er₂O₃, Sm₂O₃ and Pr203;
a near-infrared ray emitting carbon material; and
a polymer resin in which the metal oxide and the carbon material are dispersed.

2. The polymer composite of claim 1, wherein the carbon material comprises graphite.

3. The polymer composite of claim 1, wherein the carbon material has an average particle diameter of 25 to 160 nm.

4. The polymer composite of claim 1, wherein the metal oxide and carbon material are comprised in a total of 1 to 8 wt % of the total weight of the near-infrared ray emitting polymer composite.

5. The polymer composite of claim 1, wherein the polymer composite comprises 10 to 40 parts by weight of the carbon material based on 100 parts by weight of the metal oxide.

6. The polymer composite of claim 1, wherein the polymer composite comprises 67 to 88 wt % of Nd₂O₃ and Er₂O₃ based on the total weight of the metal oxide.

7. The polymer composite of claim 1, wherein the polymer composite comprises 12 to 33 wt % of Sm₂O₃ and Pr₂O₃ based on the total weight of the metal oxide.

8. The polymer composite of claim 1, wherein the metal oxide has an average particle diameter of 50 to 500 nm.

9. The polymer composite of claim 1, wherein the polymer resin comprises at least one selected from the group consisting of polyamide, polyester, polyketone, liquid crystal polymer, polyolefin, polyphenylene sulfide (PPS), polyether ether ketone (PEEK), polyphenylene oxide (PPO), polyether sulfone (PES), polyether imide (PEI) and polyimide.

10. A near-infrared ray emitting commodity, comprising the polymer composite according to claims 1.

11. The near-infrared ray emitting commodity of claim 10, wherein the near-infrared ray emitting commodity comprises at least one of clothing, a food container and a cosmetic container.

12. The near-infrared ray emitting commodity of claim 10, wherein the near-infrared ray emitting commodity is formed by the near-infrared ray emitting polymer composite, or is formed by coating the near-infrared ray emitting polymer composite on a predetermined commodity.
